(19) United States
(12) Patent Application Publication
(10) Pub. No.: US 2010/0261265 A1
(43) Pub. Date: Oct. 14, 2010

(54) METHOD AND DEVICE FOR TESTING CELL RESPONSES TO POLYMER PARTICLES IN VITRO

(76) Inventors: Hsu-Wei Fang, (US); Yi-Ching Ho, (US)

Correspondence Address: Dykema Gossett, PLLC, Suite 300 West, 1300 I Street, N.W., Washington, DC, 20005-3306, US

(21) Appl. No.: 12/421,888

(22) Filed: Apr. 10, 2009

(51) Int. Cl. C12M 1/34

(52) U.S. Cl. 435/288.4

(57) ABSTRACT

This invention provides a method and device for testing cell responses to polymer particles in vitro. Cells and culture medium are added to a 24-well plate and incubated for 24 hours to make the cells adhere to the interior bottom surface of the plate. The old medium is then drained out and fresh medium and polymer particles are added to fill up the wells. The plate was covered with a light and transparent film such as polyvinyl chloride (PVC) with small holes for ventilation and inverted carefully. The polymer particles float and contact with the cells adhered to the interior bottom surface of the plate. Then the cells are incubated for an intended period for further experiments.

### TECHNICAL FIELD

The present invention is related to a method and a device for testing cell responses to polymer particles in vitro, especially by inverting the plate to make polymer particles float and contact with the cells.

### BACKGROUND OF THE INVENTION

After an artificial implant is implanted into human body, the two contacting surfaces of the implant may be rubbed with each other during exercise, resulting in production of wear particles. The wear particles will induce a series of physiologically reaction in human body, for example, activation and aggregation of macrophages. It has been found in lots of investigations that wear particles produced by an artificial joint, after ingested by macrophages, may cause secretion of inflammatory substances. Especially the wear particles produced by ultra-high molecular weight polyethylene (UHMWPE, one of the constituting materials of artificial joins), after ingested by macrophages, may lead to secretion of the inflammatory substances which can induce activation of osteoclasts, resulting in osteolysis and in turn loosening or damaging of the artificial joint. Accordingly, it becomes very important to investigate the biological responses caused by wear particles.

Ingram etc. cultured murine macrophages in solid culture medium containing wear particles of ultra-high molecular weight polyethylene. However, not like in an in-vivo environment which is full of body fluid, polyethylene particles and murine macrophages cannot be freely moved in said solid culture medium.

Boynton etc. cultured murine macrophage cell line in a plate with ultra-high molecular weight polyethylene particles fixed on its bottom with collagen protein. As the polyethylene particles were in a fixed state, ingestion of these particles by macrophages may become less efficient.

However, on testing cell responses to polymer particles, these polymer particles, if not fixed, may float up to the upper layer of the culture medium because the density of polymer is smaller than that of common liquid mediums. This will result in insufficient contact of the polymer particles with the cells and cause troubles in performing the test. For example, the density of ultra-high molecular weight polyethylene particles is about 0.94 g/cm³, which is slightly smaller than the density of water. On testing the biological responses of the in-vitro cultured cells to ultra-high molecular weight polyethylene particles, these particles may float up to the upper layer of the culture medium and hence cannot sufficiently contact with the cells adhered to the bottom surface of the plate. This will make the test unfeasible.

So far, the technologies of testing cell responses to polymer particles such as polyethylene particles in vitro, especially in a liquid culture medium simulating in-vivo environment, are still lacking.

### SUMMARY OF INVENTION

In order to resolve the above problems, the present invention provides a method and a device for testing cell responses to polymer particles in vitro, wherein the polymer particles contact with the cells in a liquid culture medium to test cell responses to the polymer particles.

In the present invention, invert cell culture can provide a fluid culture environment similar to in-vivo environment, wherein polymer particles can move freely and contact with cells sufficiently. The present invention can help researchers to understand the mechanism of the effects of polymer particles such as polyethylene particles on cellular physiology.

The first aspect of the present invention provides a method of testing cell responses to polymer particles in vitro, comprising:
(1) providing a culture device with the cells adhered to its interior bottom surface;
(2) to the culture device, adding polymer particles and a liquid culturing medium having a density higher than that of the polymer particles;
(3) covering the open end opposite to the bottom of the culture device;
(4) inverting the culture device to bring the floating polymer particles into contact with the cells;
(5) testing the responses of the cells.

The second aspect of the present invention provides a device of testing cell responses to polymer particles in vitro, comprising a well or wells, each having

a sealed bottom,

an open end opposite to the bottom, and

a cover for covering the open end to prevent the content of the well from flowing out,

characterized in that a liquid culture medium having a density higher than that of the polymer particles is added to each well.

### BRIEF DESCRIPTION OF DRAWINGS

FIG. 1A is a schematic diagram showing the lateral view of the inverted plate according to the preferred example of the present invention.

FIG. 1B is a schematic plan view of a cover.

FIG. 2A shows the relative cell viability under different ratios of particle number to cell number.

FIG. 2B shows the relative cell viability of the upright plates and the inverted plates.

FIG. 2C shows the concentrations of cytokine TNF-α under different ratios of particle number to cell number.

### DETAILED DESCRIPTION OF PREFERRED EMBODIMENTS

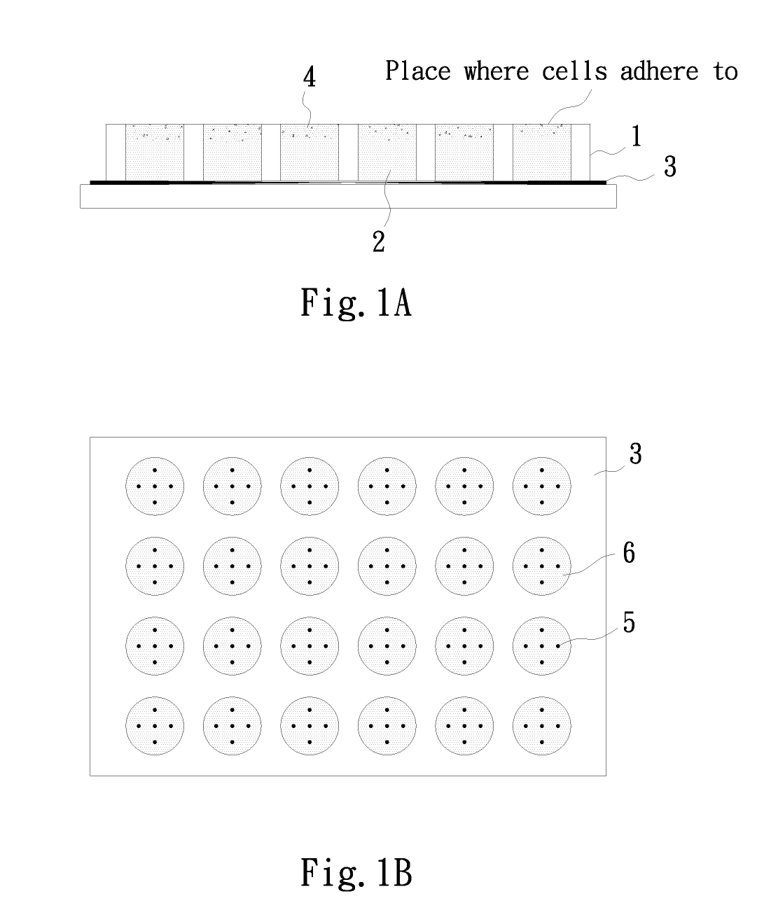

FIG. 1A is a schematic diagram showing the lateral view of the inverted plate according to the preferred example of the present invention.

As shown in FIG. 1A, a 24-well plate 1, which is used as the culture device, is filled with culture medium 2 and is covered with polyvinyl chloride film 3 with small vent holes. The plate is then inverted and the polyvinyl chloride film 3 is adhered to the open end of the plate 1 through the action of atmospheric pressure. The film 3 would not be separated from the plate even under mild shaking. The ultra-high molecular weight polyethylene particles 4 float up and become easier to contact with the cells adhered to the interior bottom surface of the plate. Moreover, as the polyvinyl chloride film 3 has small vent holes, the cells are able to receive fresh air and discharge waste gas; therefore the cells can maintain a healthy state.

The preferred embodiments according to the present invention are illustrated hereinafter.

### Example 1

### Cover Made of Polyvinyl Chloride Film

FIG. 1B is schematic plan view of a cover. As shown in FIG. 51B, polyvinyl chloride film 3 was sized to the same shape and size as the cell plate. At said film 3, five holes with a diameter of 0.1 cm were drilled at the positions 6 which correspond to the well positions of the plate, to serve as the vent hole 5. The vent hole 5 could be any size, if sufficient air could be supplied and leakage of the culture medium could be prevented. Preferably, after the polyvinyl chloride film 3 was pressed by a heavy article for several days, 15 pieces of double sided foam tape with a size of 0.3 cm×0.3 cm and a thickness of 0.5 cm were adhered to the film 3 at the spaces among the positions 6, to provide a support when the plate was inverted, so as to prevent the vent hole 5 from closing. The film 3 was then washed with acetone to remove the grease and impurities adhered to the film 3. After air dry, the film 3 was sprayed with large amount 70% alcohol and was irradiated with ultraviolet light overnight to kill the microorganisms contaminating the film 3.

### Example 2

### Suspension of Ultra-High Molecular Weight Polyethylene Particles

Ultra-high molecular weight polyethylene particles having particle size of 3.3 μm were added to a plate and were suspended in the culture medium to form a suspension of ultra-high molecular weight polyethylene particles. A small amount of the cell suspension was taken and dripped into a blood cell counting plate. The cells in the cell suspension were counted under an optical microscope, thereby determining the density of the ultra-high molecular weight polyethylene particles in the cell suspension.

### Example 3

### Cell Culture

J774A.1, a murine macrophage line, was cultured in DMEM medium containing 10% fetal bovine serum. After grown to confluence, the cells were washed with PBS one or two times, scraped off the plate with a blade and then suspended in PBS. The cell suspension was centrifuged at 1500 rpm for 10 minutes and the supernatant was removed. The cells were re-suspended in fresh medium. A small amount of the cell suspension was taken and dripped into a blood cell counting plate. The cells in the cell suspension were counted under an optical microscope, thereby determining the density of the cells in the cell suspension. One milliliter of medium and 3×105 cells were added to a 24-well plate and incubated in a incubator containing 5% CO2 at 37° C. for 24 hours. After the cells adhered to the interior bottom surface of the plate, the old medium was replaced with 3 ml of fresh medium and a suspension of ultra-high molecular weight polyethylene particles was then added to fill up the well. The number ratios of the ultra-high molecular weight polyethylene particles to the cells were adjusted to 0:1, 0.1:1 and 1:1, respectively. The plate was covered with a treated polyvinyl chloride film, and then carefully inverted. The inverted plate was incubated in an incubator containing 5% CO2 at 37° C. for 5 days.

### Example 4

### Test of Cell Viability

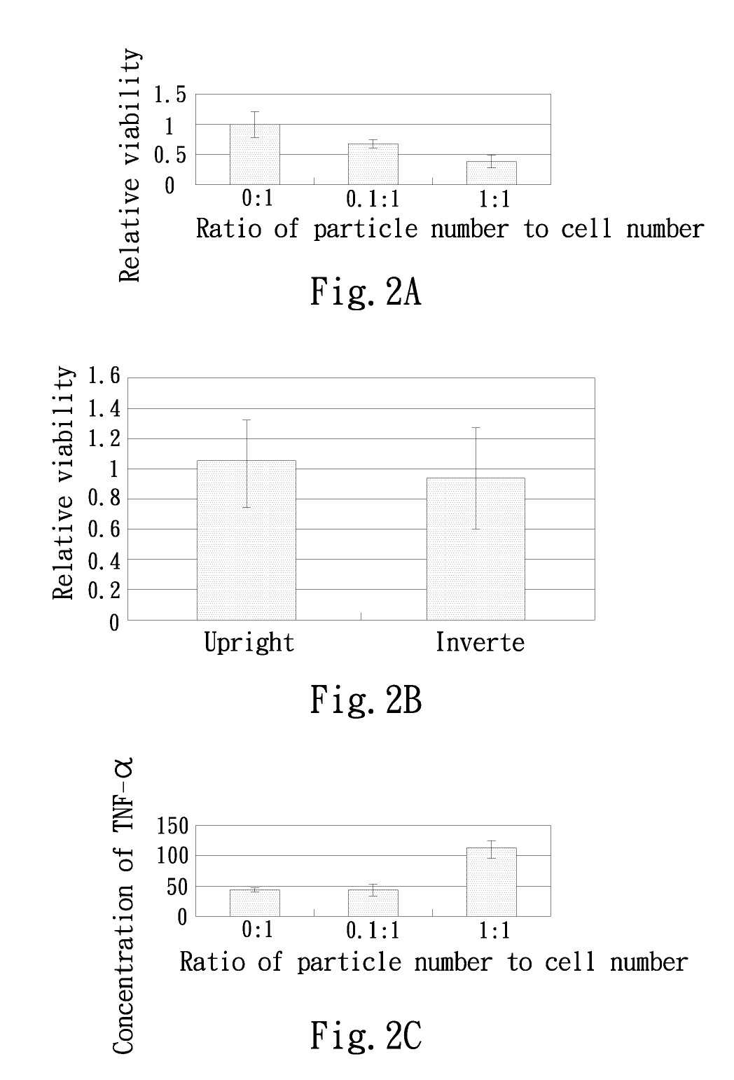

After culture was finished, the medium in the plate was drained out, then 100 μl of 0.2% MTT solution was added to each well of the plate under light-shielded condition and the plate was incubated at 37° C. for 3 hours. MTT solution was drained out and 200 μl of DMSO was added to each well. After the plate was shaken for 15 minutes, 100 μl of the sample was taken out from each well and added to an ELISA plate. The absorbance at 570 nm of the sample was determined by using an ELISA reader and the cell viability was calculated from the absorbance values. The results were shown in FIGS. 2A and 2B. FIG. 2A showed relative cell viability under different ratios of particle number to cell number. FIG. 2B showed the relative cell viability of the upright plates and the inverted plates. From these results, it can conclude that cell viability was not changed in the inverted plates.

### Example 5

### Test of Cytokine TNF-α

In this Example, the concentration of cytokine TNF-α was measured by an ELISA method. A capture antibody was added to the wells of an ELISA plate and incubated overnight. Next day, the capture antibody was drained out. After washing three times, a blocking buffer was added and incubated for 1 hour. After washing 3 times, the medium that had been used for culturing the cells was added and incubated for 2 hours. After washing 3 times, a detection antibody was added and incubated for 2 hours. After washing 3 times, streptavidin-HRP was added and incubated for 20 minutes in a dark place. After washing 3 times, a substrate solution was added and incubated for 20 minutes in a dark place. A stop solution was then added. The absorbance at 450 nm of the sample was measured using an ELISA reader and the concentration of TNF-α was calculated from the measured absorbance value. The results under different ratios of particle number to cell number were shown in FIG. 2C.

As stated above, the present invention complies with the three requirements for a patentable invention: novelty, inventive-step (non-obviousness) and industrial utilization. The present invention has been disclosed by above preferred Examples. However, the persons skilled in the art should understand that these Examples just illustrate the present invention but by no means limit the scope of the present invention in any way. It should be also noted that any modification and replacement having equivalent effects to these Examples are considered falling into the scope of the present invention, and the scope of the present invention is indicated by the following claims.

1.-5. (canceled)

6. A device that is used for testing cell responses to polymer particles in vitro, comprising a well or wells, and a liquid culture medium comprising a higher density than the polymer particles, wherein each well having
a sealed bottom;
an open end opposite to a bottom, and
a cover for covering the open end to prevent content of the well from flowing out.

7. The device of claim 6, wherein the cover is made of polyvinyl chloride film.

8. The device according to claim 6 , wherein the cover has vent holes.

9. The device according to claim 6, wherein the polymer particles are polyethylene particles.
